(19) United States
(12) Patent Application Publication
(10) Pub. No.: US 2021/0388022 A1
(43) Pub. Date: Dec. 16, 2021

(54) SUBSTITUTED GUANIDINO AND AMIDINO REAGENTS AND THE USE THEREOF FOR PROTEIN DENATURATION

(71) Applicant: Waters Technologies Corporation, Milford, MA (US)

(72) Inventors: Xiaoxiao Liu, Natick, MA (US); Wenjing Li, Shrewsbury, MA (US); Matthew A. Lauber, North Smithfield, RI (US)

(73) Assignee: Waters Technologies Corporation, Milford, MA (US)

(21) Appl. No.: 17/314,748

(22) Filed: May 7, 2021

Related U.S. Application Data
(60) Provisional application No. 63/038,366, filed on Jun. 12, 2020.

(51) Int. Cl. C07K 1/113; C07C 279/04; C12Q 1/37; C07D 487/04

(52) U.S. Cl. CPC C07K 1/1136; C07D 487/04; C12Q 1/37; C07C 279/04

(57) ABSTRACT

The present disclosure relates to a system for a composition for protein denaturation. The composition includes a non-nucleophilic denaturant comprising a substituted guanidine, wherein the denaturant has a pKa value greater than about 10, and wherein the concentration of the substituted guanidine is less than 250 mM.

### CROSS-REFERENCE TO RELATED APPLICATIONS

This application claims benefit and priority to U.S. Provisional Application No. 63/038,366, filed Jun. 12, 2020, entitled “Substituted Guanidino and Amidino Reagents and The Use Thereof for Protein Denaturation.” The content of which is incorporated herein by reference in its entirety.

### FIELD OF THE TECHNOLOGY

The present disclosure relates generally to denaturing proteins. More specifically, the present disclosure relates to using substituted guanidino and amidino reagents to disrupt protein structures.

### BACKGROUND

Ions can have a stabilizing or destabilizing effect based on whether they have an ordering, or conversely disordering, effect on water. In the case of ions that cause an increase in water coordination, protein structures are found to be stabilized. In turn, various sample preparation approaches may be needed to denature these protein structures for analysis.

### SUMMARY

Some proteins are resistant to denaturation, which has necessitated the discovery of more powerful denaturants. However, using too much of a denaturant has process drawbacks. The present disclosure describes two denaturants, substituted guanidino and amidino reagents, which are non-nucleophilic. This makes it possible to use the substituted guanidino and amidino reagents without imposing any interference with downstream derivatization reactions involving electrophilic reagents.

In some aspects, the present disclosure provides a composition for protein denaturation. The composition includes a non-nucleophilic denaturant comprising a substituted guanidine. The denaturant has a pKa value greater than about 10, and the concentration of the substituted guanidine is less than 250 mM.

In some embodiments, the substituted guanidine is selected from the group consisting essentially of tetramethylguanidine, tertbutyl tetramethylguanidine, triazabicyclodecene, or combinations thereof. In some embodiments, the substituted guanidine comprises at least one from the group of tetramethylguanidine, tertbutyl tetramethylguanidine, triazabicyclodecene, or combinations thereof. In some embodiments, the substituted guanidine of tetramethylguanidine is 1,1,3,3-tetramethylguanidine with the chemical structure of

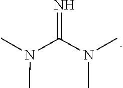

In some embodiments, the substituted guanidine of tertbutyl tetramethylguanidine is 2-tert-butyl-1,1,3,3-tetramethylguanidine with the chemical structure of

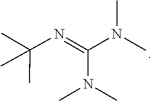

In some embodiments, the substituted guanidine of triazabicyclodecene is 1,5,7-triazabicyclo[4.4.0]dec-5-ene with the chemical structure of

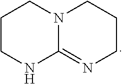

In some embodiments, the substituted guanidine is a guanidinium cation. In some embodiments, the composition further includes an additional denaturant of at least one from the group of sodium dodecylsulfate, n-lauryl sarcosine, lauric acid, cholic acid, or combinations thereof.

In some aspects, the present disclosure provides a composition for protein denaturation. The composition includes a non-nucleophilic denaturant comprising a substituted amidine. The denaturant has a pKa value greater than about 10, and the concentration of the substituted amidine is less than 250 mM.

In some embodiments, the substituted amidine is selected from the group consisting essentially of hexanimidamide, acetamidine, propanimidamide, or combinations thereof. In some embodiments, the substituted amidine comprises at least one from the group of hexanimidamide, acetamidine, propanimidamide, or combinations thereof.

In some aspects, the present disclosure provides a method of denaturing a sample comprising a protein. The method includes incubating the sample with a non-nucleophilic denaturant; heating the sample for a predetermined amount of time to denature the protein; and cooling the sample to a reduced temperature. The concentration of the denaturant is less than about 250 mM, and the denaturant has a pKa value greater than about 10.

In some embodiments, the non-nucleophilic denaturant comprises substituted guanidine, substituted amidine, or a combination thereof. In some embodiments, the substituted guanidine comprises tetramethylguanidine, tertbutyl tetramethylguanidine, triazabicyclodecene, or combinations thereof. In some embodiments, the substituted amidine comprises hexanimidamide, acetamidine, propanimidamide, or combinations thereof. In some embodiments, the denatured protein is unfolded and remains unfolded when the temperature is reduced to the reduced temperature. In some embodiments, the non-nucleophilic denaturant is selected from the group consisting essentially of tetramethylguanidine, tertbutyl tetramethylguanidine, triazabicyclodecene, or combinations thereof. In some embodiments, heating the sample comprises heating the sample to a temperature of at least 40° C. In some embodiments, heating the sample comprises heating the sample to a temperature ranging from about 40° C. to about 100° C. In some embodiments, the reduced temperature ranges from about 30° C. to 75° C. In some embodiments, the method includes diluting the cooled sample and/or digesting the cooled sample. In some embodiments, the method includes digesting the sample with a protease. In some embodiments, the protease is trypsin, Lys-C, Arg-C, Glu-C, Asp-N, chymotrypsin, or combinations thereof. In some embodiments, the method includes treating the cooled sample with an endo or exoglycosidase.

The present disclosure provides many advantages including using substituted guanidino and amidino reagents, which are non-nucleophilic, as denaturants without imposing any interference with downstream derivatization reactions involving electrophilic reagents. While not wishing to be bound by theory, it is reasonable to suggest that substituted guanidino and amidino reagents are unique in their ability to strongly ion pair to anionic protein sites and to simultaneously introduce hydrophobicity to the local microenvironment of a protein domain. This amphipathic property is believed to disrupt the solvation of the ion paired protein domain such that entropy no longer favors it to be folded in its native structure. These substituted guanidino and amidino reagents might be particularly advantageous for achieving complete denaturation of acidic structures while being sufficiently amphipathic to converge into a micelle system, which can be inherently disruptive to protein structure. The substituted guanidino reagents can be effectively used with temperature cycling and small dilution factors to take a sample from a harshly denaturing condition to one that is only partially denaturing such that an enzyme could be readily employed. And the substituted amidino reagents can potentially lend sufficient denaturation power to high temperature sample preparation steps and then be sufficiently mild at lower temperatures so as to not interfere with a subsequent enzymatic reaction.

### BRIEF DESCRIPTION OF THE DRAWINGS

The technology will be more fully understood from the following detailed description taken in conjunction with the accompanying drawings, in which:

FIG. 1 is a flowchart of an example of a digestion workflow.

FIG. 2 displays protein denaturation as observed through a change in the native fluorescence of rabbit IgG as a function of guanidine hydrochloride concentration, in accordance with the present disclosure.

FIG. 3 displays fluorescence emission intensity ratios of rabbit IgG after incubation with different substituted guanidine reagents (Reagent 1, 2 and 3) at varying temperature, in accordance with the present disclosure.

FIG. 4 displays fluorescence emission intensity ratios of rabbit IgG after incubation with different substituted amidino reagents (Reagent 4, 5 and 6) at varying temperature, in accordance with the present disclosure.

FIGS. 5A-5E display reversed phase LC-MS chromatograms of intact protein RPLC of Orencia® (abatacept, available from Bristol-Myers Squibb) before and after denaturation with various reagents and PNGase F deglycosylation, in accordance with the present disclosure.

FIG. 6 displays fluorescence resulting from a mixed mode separation of RapiFluor-MS™ labeled (commercially available from Waters Technologies Corporation, Milford Mass.) N-glycans derived from Orencia® and denaturation with reagent #1 (1,1,3,3-tetramethylguanidine), in accordance with the present disclosure.

FIGS. 7A-7D are graphs displaying 1 mM of each tetramethylguanidine denaturants (FIG. 7A is a blank; Reagent #1, FIG. 7B; Reagent #2, FIG. 7C; and Reagent #3, FIG. 7D) in digestion buffer to analyze peptide mapping through a generic LC-MS method, in accordance with the present disclosure.

FIG. 8 is a table displaying chemical names and structures of substituted guanidine and amidine reagents for protein denaturation.

### DETAILED DESCRIPTION

Some proteins are resistant to denaturation, which has necessitated the discovery of more powerful denaturants. Two denaturants are substituted guanidino and amidino reagents, which are non-nucleophilic. This makes it possible to use the substituted guanidino and amidino reagents without imposing any interference with downstream derivatization reactions involving electrophilic reagents.

Guanidine derivatives comprised of varying substituents are shown to be powerful effectors of protein structure. As denaturants, it has been found that these compounds can disrupt common protein structures at sub-millimolar concentrations. In some embodiments, this denaturation power is used advantageously to unfold recalcitrant tertiary and quaternary protein structure and prepare them for enzymatic processing, such as protein digestion or glycan release.

Substituted guanidino reagents include where one or more N—H of the reagent is replaced with an alkyl, aryl, cyclo, heteroatom containing, alkene, alkyne, PEG, PEO, etc. moiety. In some examples, the substitutions can be interconnected to form cyclic rings. The same applies to substituted amidino reagents. One but not necessarily all N—H are substituted to a non-hydrogen functionality. Substituted amidino reagents can alternatively be used as options to achieve milder, more enzyme-friendly denaturation. Both the substituted guanidino and amidino reagents can be combined with derivatization reactions involving the electrophilic reagents and nucleophilic substitution reactions.

The concentration of the substituted guanidino reagents and substituted amidino reagents, when described herein, is referring to the buffered solution. The buffered solution can include common buffers and ionic strength adjusting salts. Common buffers include phosphate, tris(hydroxymethyl)aminomethane, tris bis propane, triethyl amine, and other common buffers. Common ionic strength adjusting salts include NaCl, KCl, Ca²⁺, or other divalent or monovalent cations and anions.

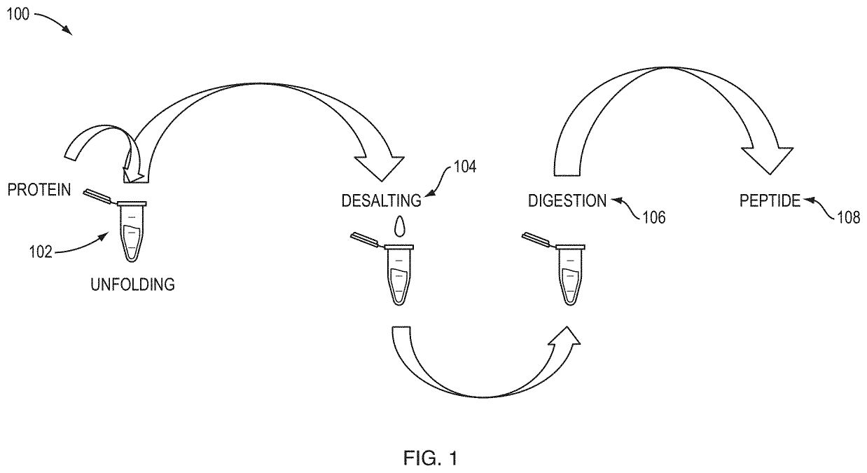

FIG. 1 is a flow chart illustrating an overview of the peptide mapping workflow 100. In some examples, peptide mapping workflow 100 includes four parts. A part one 102 includes a sample with an analyte of interest, such as a protein, that is unfolded. Proteins vary in the difficulty of unfolding. In some examples, substituted guanidino reagents and substituted amidino reagents can be used to help unfold the proteins. A part two 104 includes desalting the sample, which includes the unfolded analyte of interest. Here, desalting devices and pressure-resistant sizing media can be used to desalt the sample. A part three 106 includes digesting the analyte of interest of the sample. For example, the desalted sample can be digested by an enzyme, which can be immobilized, such as, an immobilized protease or immobilized glycosidase. After the analyte of interest is digested, a part four 108 includes collecting the sample with digested analyte of interest.

Part one 102 and part two 104 can be considered pre-treatment steps. Part one 102 and part two 104 can be dependent on the analyte of interest. In some examples, proteins that can be easily denatured by heat and are introduced during digestion do not require pretreatment. For proteins that need pretreatment, denaturation followed with reduction and alkylation are common steps to fully unfold the protein. After part one 102 where the protein of the sample is unfolded, part two 104 is often required to desalt the sample. Besides proteins, the analyte of interest can be a nucleic acid, nucleoprotein complex, peptide, or viral particles.

Guanidine and sodium dodecyl sulfate (SDS) have long been used to denature proteins. In turn, they have become ubiquitous in various sample preparation approaches where complete denaturation is needed in order to achieve accurate and precise analyses.

Ions can have stabilizing or destabilizing effects based on whether they have an ordering or conversely disordering effect on water. In the case of ions that cause an increase in water coordination, protein structure is found to be stabilized. These types of ions are referred to as kosmotropes. Conversely, a set of ions, known as chaotropes, disrupt ordered water and thereby destabilize proteins by minimizing the entropic force that stabilizes them. One of the most effective and widely used chaotropes is guanidine. In the form of a guanidinium ion, this reagent is capable of abolishing most protein structures. However, it is sometimes necessary to use guanidine at high concentrations, such as concentrations exceeding 6M. These high concentrations can require samples to be extensively desalted prior to enzymatic sample preparation steps, such as protein digestion and deglycosylation.

The surfactant properties of SDS can be taken advantage of to achieve denaturation. In most cases, denaturation is achieved with a surfactant only through the combined use of a high temperature incubation (e.g., >70° C.). Proteins are usually boiled with SDS. Upon being unfolded, the protein is stabilized in its denatured state by the amphipathic nature of the SDS molecule. The sulfo head group of the SDS ion pairs with basic amino acid residues while the lipophilic tail interacts with the more hydrophobic portions of the denatured structure. It is this property of SDS that helps facilitate size-based gel electrophoresis separations.

Unfortunately, most surfactants like SDS are too hydrophobic to be tolerated by other common protein characterization techniques, e.g., C18 reversed phase chromatography. And C18 reversed phase chromatography is used for peptide mapping.

Therefore, a need exists for alternative reagents for achieving protein denaturation that facilitate complete denaturation and are not deleterious to subsequent enzymatic reactions, derivatization reactions, or downstream chromatography.

The present disclosure provides substituted guanidines as novel reagents to use with heat-activated protein denaturation and subsequent protein digestion and protein deglycosylation steps. Substituted guanidino reagents include where one or more N—H of the reagent is replaced with an alkyl, aryl, cyclo, heteroatom containing, alkene, alkyne, PEG, PEO, etc moiety. In some examples, the substitutions can be interconnected to form cyclic rings. The same applies to substituted amidino reagents. One but not necessarily all N—H are substituted to a non-hydrogen functionality. As described herein, guanidines are a group of compounds sharing the general structure (R₁R₂N)(R₃R₄N)C═N—R.

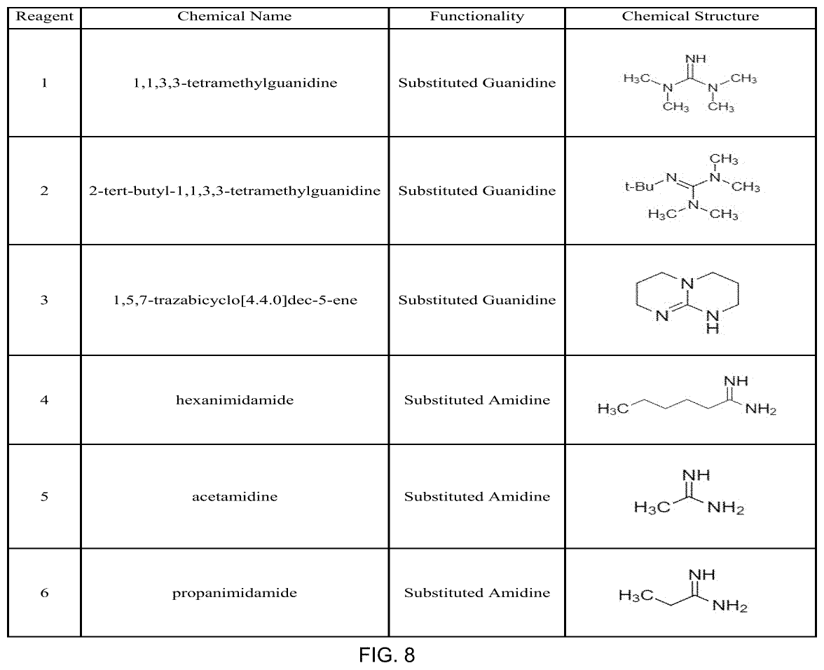

In their conjugate acid form, guanidines are present as guanidinium cations, which are planar, symmetric ions bearing a highly stable 1+charge. The resonance stabilization of the charge results in efficient solvation by water and high pKa values that are generally greater than 12. In neutral aqueous solutions, guanidines exists almost exclusively as guanidinium cations. Three example substituted guanidines include but are not limited to tetramethylguanidine, tertbutyl tetramethylguanidine, and trazabicyclodecene (FIG. 8). FIG. 8 provides a table showing chemical structures in the deprotonated state, although the reagents in the present disclosure have been used in their protonated form as prepared in a buffered solution.

The present disclosure also provides substituted amidines as novel reagents for protein denaturation. A substituted amidine is defined herein with the general structure (R₁R₂N)(R₃)C═N—R. Three examples of an amidine reagent include, but are not limited to, hexanimidamide, acetamidine, and propanimidamide (FIG. 8). Like guanidine reagents, amidines are strongly basic (pKa>12) as a result of the resonance stabilization they exhibit when protonated. In neutral solution, amidines are present in their protonated amidine form. Both guanidine and amidine reagents are non-nucleophilic, which is advantageous if they are to be used with electrophilic derivatization reagents like RapiFluor-MS™ (available from Waters Technologies Corporation, Milford, Mass.).

In some examples, part one 102 can be a method of denaturing a sample including an analyte of interest, such as a protein. The method can include incubating the sample with a non-nucleophilic denaturant, heating the sample for a predetermined amount of time to denature the protein, and cooling the sample to a reduced temperature. The buffered solution concentration of the denaturant can be less than about 250 mM and the denaturant can have a pKa value greater than about 10. The non-nucleophilic denaturant can include substituted guanidine, substituted amidine, or a combination thereof.

Heating the sample includes heating the sample to a temperature ranging to at least 40° C. or from about 40° C. to about 100° C. The denatured protein of the sample is unfolded and remains unfolded when the temperature is reduced to the reduced temperature. The reduced temperature of the cooled sample can be from about 30° C. to 75° C. The cooled sample can be further diluted and/or digested.

Digesting the sample can include digesting the sample with a protease. In some examples, the protease includes trypsin, Lys-C, Arg-C, Glu-C, Asp-N, chymotrypsin, or combinations thereof. The cooled sample can also be treated with an endo or exoglycosidase. The glycosidase can be Peptide-N-glycosidase F (PNGase F), EndoS, EndoS2, OpeRATOR™ (O-glycan-specific protease) (available from Genovis Inc., Cambridge, Mass.), GlycOCATCH® (enrichment resin for O-glycosylated proteins and peptides) (available from Genovis Inc., Cambridge, Mass.), OglyZOR® (O-glycosidase) (available from Genovis Inc., Cambridge, Mass.), and SialEXO® (sialidase) (available from Genovis Inc., Cambridge, Mass.).

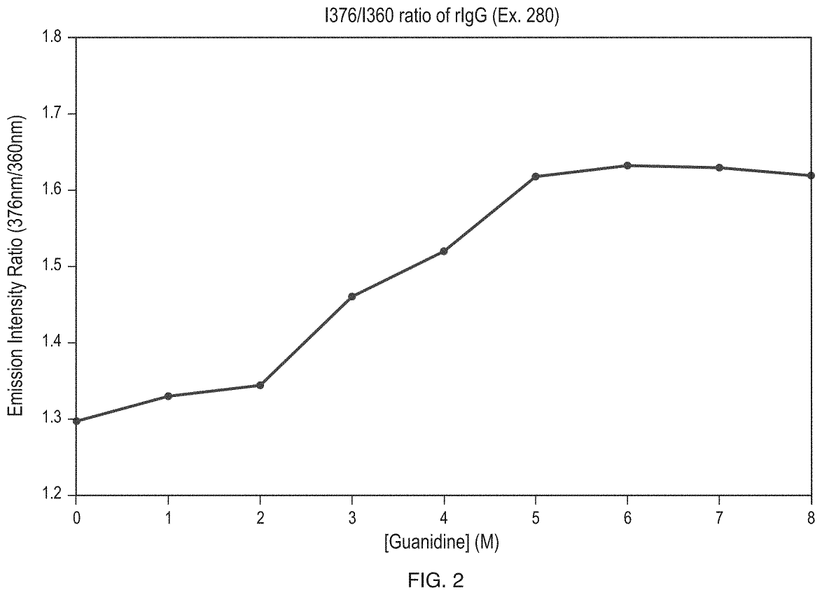

FIG. 2 displays protein denaturation as observed through a change in the native fluorescence of rabbit IgG as a function of guanidine hydrochloride concentration. To determine the effectiveness of the guanidine and amidine reagents from the table of FIG. 8, a native fluorescence assay was established using rabbit IgG. Upon excitation at 280 nm, emission was measured ratiometrically at two wavelengths 376 and 360 nm. As seen in FIG. 2, this approach allowed for the generation of denaturation curve for subjecting the sample to increasing concentrations of guanidine.

Fluorescence spectra were acquired at an excitation wavelength of 280 nm and emission intensity is reported as a ratio of 376 nm intensity divided by 360 nm intensity. An increase in 1376/1360 denotes a change in the microenvironment of tryptophan residues—specifically, their change from a hydrophobically surrounded native state to a solvent exposed denatured state.

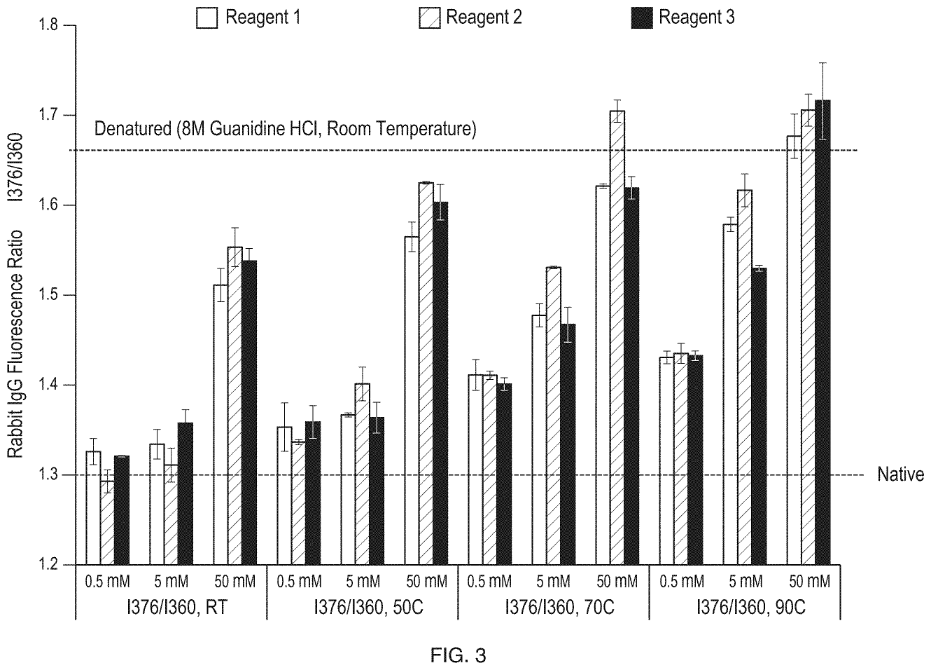

The IgG sample was subjected to concentrations of 0.5, 5, and 50 mM and temperatures of either ambient, 50° C., 70° C., or 90° C. With this approach, the six reagents shown in FIG. 8 were tested. FIG. 3 displays fluorescence emission intensity ratios of rabbit IgG after incubation with different substituted guanidine reagents at room temperature (RT), 50° C., 70° C., and 90° C., including intensity ratios resulting from incubation with 8M guanidine at room temperature (a historical benchmark for denaturation). Fluorescence was performed with an excitation wavelength of 280 nm.

As shown in FIG. 3, 50 mM concentrations of reagents 1, 2, and 3 proved to be highly denaturing. With incubation at room temperature, reagents 1, 2 and 3 produced IgG samples with excitation intensity ratios ranging from 1.5 to 1.55, which is indicative of a partially denaturing effect. With an incubation temperature of 50° C., the reagents yielded increased intensity ratios ranging from 1.55 to 1.6. And with an incubation temperature of 70° C. and 90° C., excitation intensity ratios increased to values between 1.62 to 1.72, which is indicative of complete denaturation.

FIG. 3 also shows that a concentration of 5 mM is still quite effective in denaturing the IgG protein, at least when combined with a 70° C. or 90° C. incubation. At a 5 mM concentration and lower temperatures, the denaturing power of reagents 1, 2, and 3 is significantly reduced.

These results show several interesting properties about the denaturing capabilities of tetramethylguanidine (reagent #1), t-butyl tetramethylguanidine (reagent #2), and trazabicyclodecene (reagent #3). First, the data demonstrate that reagents #1, #2, and #3 are potent denaturants, as evidence by their effectiveness at room temperature with concentrations of only 50 mM. Secondly, reagents #1, #2, and #3 can be effectively used with temperature cycling and small dilution factors to take a sample from a harshly denaturing condition to one that is only partially denaturing such that an enzyme could be readily employed.

In one example, tetramethylguanidine is used at a concentration ranging from 0.1 to 250 mM, or from 0.5 to 100 mM, and an optional incubation at greater than 50° C. In some examples, t-butyl tetramethylguanidine or trazabicyclodecene are used as denaturants.

The denaturation with the substituted guanidine can be followed with a 2 to 10-fold dilution so as to lend a more enzyme friendly reaction condition. The diluted sample is thereafter digested with a protease including, but not limited to, trypsin, Lys-C, Arg-C, Glu-C, Asp-N, and chymotrypsin. The diluted sample might also be subjected to treatment with a glycosidase, including but not limited to PNGase F.

In some examples, tetramethylguanidine, t-butyl tetramethylguanidine, or trazabicyclodecene is applied to a protein at a greater than 50 mM concentration. The increase in concentration might be needed to denature the most recalcitrant protein structures.

Interestingly, substituted amidino reagents did not show themselves to be as effective at denaturing the IgG test sample as substituted guanidine reagents. Nevertheless, substituted amidino reagents have value as mild denaturants.

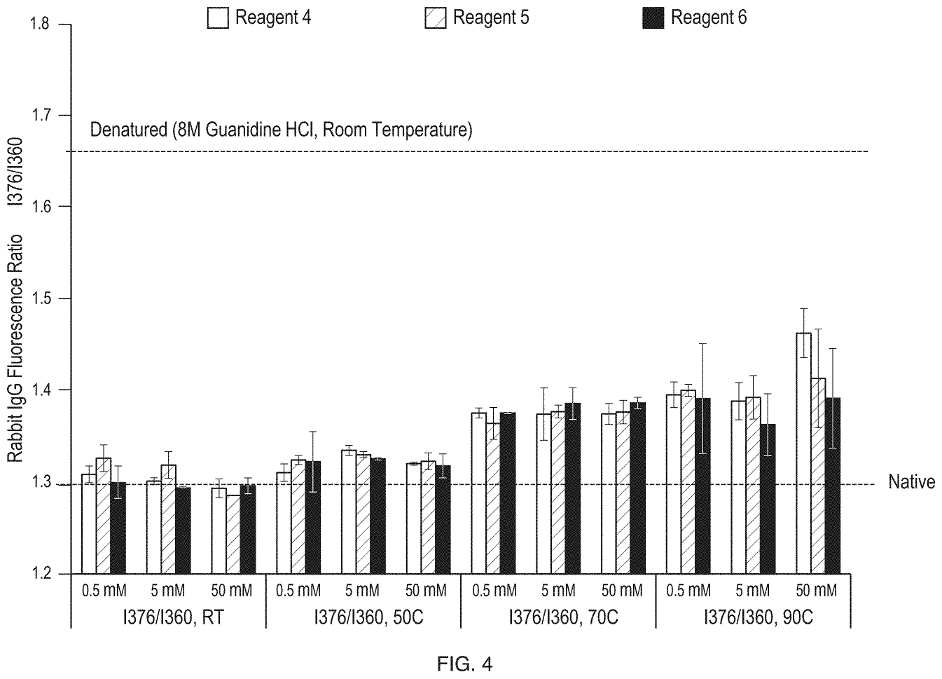

Three example substituted amidino reagents (hexanimidamide, acetamidine, propanimidamide) were tested for their denaturation effects on rabbit IgG, as shown in FIG. 4.

FIG. 4 displays fluorescence emission intensity ratios of rabbit IgG after incubation with different substituted amidino reagents at room temperature (RT), 50° C., 70° C., and 90° C., including intensity ratios resulting from incubation with 8M guanidine at room temperature (a historical benchmark for denaturation) are displayed as a reference. Fluorescence was performed with an excitation wavelength of 280 nm.

Little to no observable denaturation was found with the use of the amidino reagents even with concentrations up to 50 mM and heat denaturation temperatures up to 50° C. However, when used at a 50 mM concentration and combined with a 60° C. to 90° C. denaturation temperature, the amidino reagents were seen to be capable of inducing partial denaturation. In fact, the extent of their denaturation with such temperatures was observed to be about comparable to RapiGest™ SF surfactant (available from Waters Technologies Corporation, Milford, Mass.).

Like RapiGest™ SF surfactant, the amidino reagents could potentially lend sufficient denaturation power to high temperature sample preparation steps and then be sufficiently mild at lower temperatures so as to not interfere with a subsequent enzymatic reaction. Thus, in some examples, a substituted amidino reagent is used in place of one of the substituted guanidino reagent, particularly if an easily denatured enzyme is to be employed.

While not wishing to be bound by theory, it is reasonable to suggest that substituted guanidino and amidino reagents are unique in their ability to strongly ion pair to anionic protein sites and to simultaneously introduce hydrophobicity to the local microenvironment of a protein domain. This amphipathic property is believed to disrupt the solvation of the ion paired protein domain such that entropy no longer favors it to be folded in its native structure.

Because of the ion pairing effects of substituted guanidino and amidino reagents, these substituted guanidino and amidino reagents might be particularly advantageous for achieving complete denaturation of acidic structures, such as a protein domain that is extensively modified with sialic acid containing glycans or phosphorylated post-translational modifications. Alternatively, it is also possible that the substituted guanidino and amidino reagents are sufficiently amphipathic to converge into a micelle system, which can be inherently disruptive to protein structure.

In practice, the substituted guanidino or amidino reagents can be used alongside another denaturant including, but not limited to, sodium dodecylsulfate, n-lauryl sarcosine, lauric acid, RapiGest™ SF, ProteaseMAX™ (available from Promega Corporation, Madison, Wis.), negative ion surfactants (examples of negative ion surfactants are generally available from Protea Biosciences Group, Inc., Morgantown, W. Va.), bile salts like cholic acid, or combinations thereof.

Likewise, the substituted guanidino or amidino reagents can be used with or without heating steps. In addition, the substituted guanidine and amidine reagents presented herein can be used both with and without a desalting step prior to enzymatic reactions.

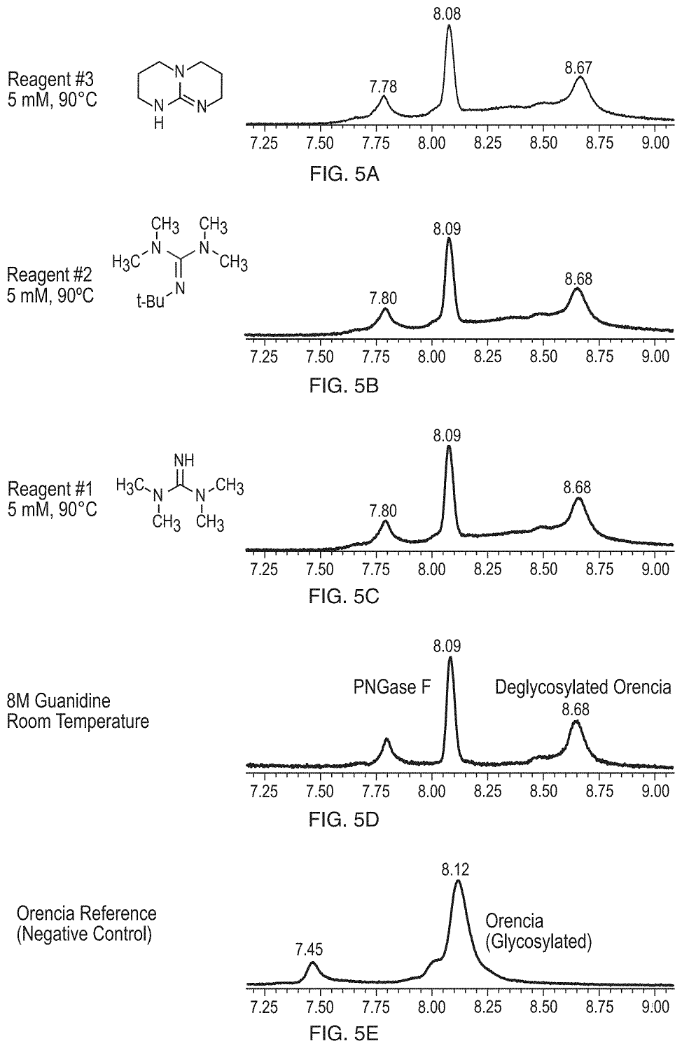

FIGS. 5A-5E display reversed phase LC-MS chromatograms of intact protein RPLC of Orencia® (abatacept, available from Bristol Myers Squibb Company) before and after denaturation with various reagents and PNGase F deglycosylation. As discussed herein, the reagents of the present disclosure can be used to facilitate protein deglycosylation. To this end, the utility of trazabicyclodecene (reagent #3, FIG. 5A), t-butyl tetramethylguanidine (reagent #2, FIG. 5B), and tetramethylguanidine (reagent #1, FIG. 5C) can be used to denature rabbit IgG and to thereafter facilitate a PNGase F deglycosylation reaction. Abatacept, a glycosylated fusion protein, was subjected to different denaturation steps and was subjected to PNGase F deglycosylation.

FIG. 5D displays a positive control sample, where abatacept was treated with 8M guanidinium hydrochloride at room temperature, desalted by size filtration, and then treated with glycosidase, which led to a new, more strongly retained peak for abatacept at 8.68 minutes. FIG. 5E displays a negative control that produced a peak for unmodified abatacept at approximately 8.12 minutes.

For FIG. 5D, that this new peak was more strongly retained suggests that the hydrophilic N-glycans of the protein were successfully cleaved. Moreover, mass spectral data supported the identification of this peak as the N-deglycosylated form of abatacept.

For comparison, trazabicyclodecene (reagent #3, FIG. 5A), t-butyl tetramethylguanidine (reagent #2, FIG. 5B), and tetramethylguanidine (reagent #1, FIG. 5C) were each individually applied to abatacept at a 5 mM concentration and a short 90° C. incubation. Upon cooling, samples were then incubated with PNGase F, and the resulting samples were found to be as extensively deglycosylated as the guanidinium hydrochloride denatured controls. These results accordingly show the effectiveness of trazabicyclodecene (reagent #3, FIG. 5A), t-butyl tetramethylguanidine (reagent #2, FIG. 5B), and tetramethylguanidine (reagent #1, FIG. 5C) to denature a glycoprotein and to facilitate the activity of a glycosidase.

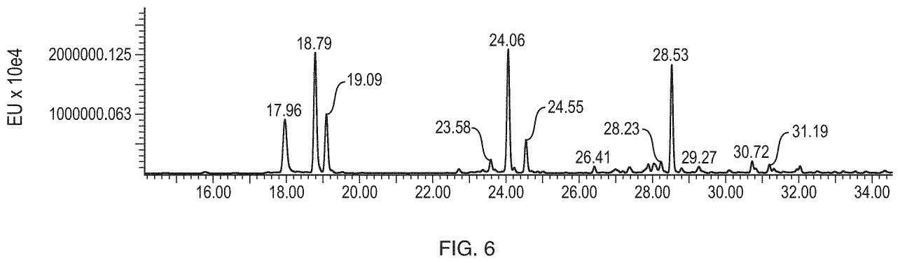

FIG. 6 displays fluorescence resulting from a mixed mode separation of RapiFluor-MS™ RapiFluor-MS™ (available from Waters Technologies Corporation, Milford, Mass.) labeled N-glycans derived from Orencia® (abatacept) and denaturation with Reagent #1 (1,1,3,3-tetramethylguanidine). In the example of FIG. 6, abatacept was denatured with 5 mM tetramethylguanidine at a 90° C. and incubated at 50° C. with PNGase F. The resulting N-glycosylamines were there derivatized with RapiFluor-MS™ and cleaned up with solid phase extraction using a GlycoWorks RapiFluor-MS™ N-glycan kit (according to manufacturer recommendations; Waters Technologies Corporation, Milford, Mass.). RapiFluor-MS™ labeled N-glycans from this tetramethylguanidinium denatured sample were thereafter profiled by anion exchange reversed phase chromatography.

FIG. 6 is a demonstration of how desalting was not required to achieve N-deglycosylation of abatacept. The 5 mM reagent concentration did not cause any significant interference to the PNGase F reactivity during its 50° C. incubation. Because the described guanidino and amidino reagents are non-nucleophilic, their use can also be directly combined with derivatization reactions entailing electrophilic reagents and nucleophilic substitution reactions. When N-glycans are cleaved from glycoproteins by PNGase F, they are first released into solution in the form of N-glycosylamines, which can be quickly derivatized with electrophilic reagents, including but not limited to RapiFluor-MS™, Instant AB (Prozyme/Agilent, Santa Clara, Calif.), Instant Pc (Prozyme/Agilent, Santa Clara, Calif.). In turn, the N-glycosylamines are imparted with a label that enhances their detectability. If a nucleophilic compound were to be added to the sample, a desalting step might be required to reduce interference with the labeling reaction.

However, in some examples, it is desirable to be able to proceed from an N-deglycosylation sample preparation step straight into a derivatization reaction so that overall sample preparation time is minimized. Thus, non-nucleophilic denaturants, like the substituted guanidino and amidino reagents, are used to help minimize overall sample preparation time. A high level of signal and a diversity of glycan species was observed for the sample of FIG. 6, which demonstrates the effectiveness of the approach. Accordingly, in some examples, the substituted guanidino or amidino reagent can be used to denature a glycoprotein that is thereafter deglycosylated and treated with an electrophilic derivatization reagent.

Guanidine, SDS and RapiGest™ SF (available from Waters Technologies Corporation, Milford, Mass.) each provide alternative mechanisms for protein denaturation. Additionally, quaternary and tertiary ammonium cations may be able to provide similar denaturation effects.

### EXEMPLIFICATION

### Example 1: Protein Denaturation Effects as Measured by Native Fluorescence Spectroscopy

Polyclonal rabbit IgG (rIgG) was used as a test protein to evaluate the denaturation power of various reagents. The rIgG protein was dissolved and diluted to a concentration of 0.25 mg/mL with water, while also being subjected to the chemical compound of interest and an incubation at either room temperature, 50° C., 70° C., or 90° C. To generate a positive control for complete denaturation, the rIgG sample was subjected to room temperature incubations with guanidine hydrochloride at 0 and up to 8M concentrations. Native fluorescence of the resulting sample was thereafter measured using an excitation wavelength of 280 nm. Emission intensities at 376 and 360 nm were subsequently detected and reported as a ratio as a sensitive measure of rIgG denaturation and a change in the local environment of tryptophan residues. These data are displayed in FIG. 2 and are representative of a classical denaturation curve. The fluorescence intensity ratio observed for native state rIgG was determined to be approximately 1.3, while that of the 8M guanidine denatured state was observed to be approximately 1.6. Meanwhile, the fluorescence intensity ratio of rIgG after being treated with 1% (w/v) RapiGest SF [˜24 mM] was seen to be approximately 1.4, which suggests it produces only partial denaturation.

Fluorescence intensity ratios obtained after incubation with reagent #1, reagent #2, and reagent #3 and either ambient, 50° C., 70° C., or 90° C. temperatures are provided in FIG. 3. Likewise, fluorescence intensity ratios obtained after incubation with reagent #4, reagent #5, and reagent #6 and either ambient, 50° C., 70° C., or 90° C. temperatures are provided in FIG. 4. In all experiments, the substituted guanidine and amidines were titrated to a neutral pH and their protonated ionization states prior to being incubated with protein sample.

### Example 2: Protein Denaturation and Subsequent Deglycosylation

To demonstrate the compatibility of using substituted guanidino denaturation with enzymatic deglycosylation with PNGase F, Orencia® (abatacept), a glycosylated fusion protein, was subjected to several different tests. An Orencia® stock solution (20 mg/mL) was diluted 10 times with water to a concentration of 2 mg/mL, followed by the addition of 10 μL diluted sample into three 1 mL polypropylene tubes. Four (4) μL of 50 mM guanidino reagents, tetramethylguanidine (reagent #1), t-butyl tetramethylguanidine (reagent #2), and trazabicyclodecene (reagent #3), were added into each tube, separately. Finally, 8 μL of 50 mM HEPES, pH 7.9 buffer was transferred into each tube along with 16.4 μL of water.

The contents of each well were subsequently mixed by aspiration. Each sample was then incubated at 90° C., 3 minutes for denaturation and then removed from the heating block to cool at room temperature for 3 minutes. Each denatured glycoprotein sample was treated with 1.6 μL of GlycoWorks Rapid PNGase F at 50° C. for 5 minutes. Aliquots containing 10 μL of each deglycosylated sample were submitted for intact protein analysis, while the remaining 30 μL samples were subjected to RapiFluor-MS™ labeling and released glycan analysis (see details in Example 3).

Additionally, two controls were applied. For a positive control (FIG. 5D), 5 μL of Orencia® stock solution was mixed with 45 μL of 8M guanidine hydrochloride at room temperature, followed by desalting with a Thermo Zeba spin column. The resulting, 10 μL desalted sample was mixed with 8 μL of 50 mM HEPES, pH 7.9, 20.4 μL of water and 1.6 μL of GlycoWorks Rapid PNGase F before being incubated at 50° C. for 5 min to perform deglycosylation. Ten (10) μL of this deglycosylated protein, as well as 10 μL of 2 mg/mL Orencia® solution without denaturation, were tested as positive and negative controls of intact protein analysis. LCMS settings and parameters used for intact protein analysis are listed below in Table 1.

TABLE 1
LCMS settings for intact protein analysis of deglycosylated abatacept

| System: | ACQUITY UPLC ® H-Class Bio System (available from Waters | | | |
|---|---|---|---|---|
| | Technologies Corporation, Milford, MA) [Consisting of a QSM | | | |
| | with 100 μL Mixer, TUV Detector (Flow cell: 500 nL Analytical), | | | |
| | FTN-SM, and CH-A heater] [Post-column tubing to FLR: 0.0025” | | | |
| | ID PEEK, 8.5” Length (p/n 700009971)] coupled to a Xevo ® G2- | | | |
| | XS QTof Mass Spectrometer (available from Waters Technologies | | | |
| | Corporation, Milford, MA) | | | |
| Data Acquisition | MassLynx ™ 4.1 (available from Waters Technologies | | | |
| and Analysis: | Corporation, Milford, MA) | | | |
| Column: | BioResolve ™ RP (available from Waters Technologies | | | |
| | Corporation, Milford, MA) mAb Polypenyl, 2.7 μm, 450 Å 2.1 × 50 mm | | | |
| Column Temperature: | 80° C. | | | |
| Seal Wash: | 10% HPLC grade Acetonitrile/90% HPLC grade water v/v (Seal | | | |
| | Wash interval set to 0.5 min) | | | |
| Sample Manager Wash: | HPLC grade water | | | |
| Mobile Phase A: | 0.1% TFA in 18.2 MΩ HPLC grade water | | | |
| Mobile Phase B: | 0.1% TFA in Acetonitrile | | | |
| Flow Rate: | 0.5 mL/min | | | |
| Gradient: | Time (min) | % A | % B | Curve |
| | 0.00 | 85.0 | 15.0 | 6 |
| | 10.00 | 55.0 | 45.0 | 6 |
| | 11.00 | 20.0 | 80.0 | 6 |
| | 11.50 | 20.0 | 80.0 | 6 |
| | 11.51 | 85.0 | 15.0 | 6 |
| | 15.00 | 85.0 | 15.0 | 6 |

| | |
|---|---|
| Sample Temperature | 10° C. |
| Samples: | 10 μL of deglycosylated Orencia ® (freshly prepared from about 5 |
| | μg glycoprotein) |
| Sample dilution: | 10 μL, of 0.1% FA |
| Sample Injection Volume: | 5 μL |
| TUV Wavelength: | 280 nm |
| Sampling Rate: | 20 points/second |
| Time Constant: | 0.1 second |
| MS Capillary Voltage: | 3.0 kV |
| Cone Voltage: | 190 V |
| Source Offset: | 80 V |
| Source Temperature: | 150° C. |
| Desolvation Temperature: | 500° C. |
| Desolvation Gas Flow Rate: | 1000 L/Hr |
| Calibration | Sodium Iodide, 100-3000 m/z |
| Acquisition: | 500-5000 m/z |
| Scan time: | 0.1 second |

As shown in FIG. 5E, the unmodified Orencia® reference (negative control) produced a broad peak around 8.12 minutes in a reversed phase separation, which corresponds to protein with N-glycan heterogeneity. While in the chromatogram of positive control, a newly formed peak appeared at a retention time of 8.68 minutes, indicating that N-glycans were successfully removed from the protein after enzymatic deglycosylation, as a result of reduced hydrophilicity and stronger retention. The presence of N-deglycosylated protein was confirmed by MS analysis. The peak around 8.09 minutes in the positive control was identified to be the remaining PNGase F in the sample.

Notably, the reversed phase chromatograms of substituted guanidino treated samples (FIGS. 5A-5C) are seen to be highly similar to that of the positive control (FIG. 5D), which suggests the subsequent deglycosylation of protein denatured with 5 mM guanidino and heat incubation was as efficient as using a traditional 8 M guanidine hydrochloride treatment. Moreover, with the benefit of temperature dependent, strongly denaturing effects of substituted guanidino reagents, an extra desalting step could be eliminated without causing negative impact to the activity of the PNGase F glycosidase, since a lower temperature is usually applied for enzymatic digestion.

### Example 3: Use of Substituted Guanidine Denaturant with N-Glycan Release, Labeling and Analysis

To demonstrate that the aforementioned substituted guanidines can be used as supplemental denaturants for N-glycan release and rapid labeling with a RapiFluor-MS™ GlycoWorks kit (available from Waters Technologies Corporation, Milford, Mass.), N-glycan release and labeling was performed on deglycosylated Orencia® samples saved from intact protein analysis (see details in Example 2). Thirty (30) μL of deglycosylated protein was labeled with 12 μL of RapiFluor-MS™ solution (68.7 mg/mL in DMF) for 5 minutes at room temperature, followed by dilution with 358 μL of acetonitrile for SPE clean-up. The total of 400 μL mixed solution was transferred to a pre-conditioned HILIC μElution SPE plate staged for operation on a positive pressure manifold with 3 psi pressurization. Each sample was loaded onto the plate and then twice washed with 600 μL of 1:9:90 (v/v/v) formic acid/water/acetonitrile. Finally, released glycans were eluted from individual wells with three, 30 μL of 200 mM ammonium acetate in 5% acetonitrile. Eluate was collected and transferred in LC vials for analysis. LC-FLR-MS settings and parameters used in these experiments are listed below in Table 2.

TABLE 2
LC-FLR-MS settings of N-Glycans released from Orencia ® and labeled with RapiFluor-MS

| System: | ACQUITY UPLC ® H-Class Bio System (available from Waters | | | |
|---|---|---|---|---|
| | Technologies Corporation, Milford, MA) [Consisting of a QSM | | | |
| | with 100 μL Mixer, FLR Detector (Flow cell: 1000 nL Analytical), | | | |
| | FTN-SM, and CH-A heater] [Post-column tubing to FLR: 0.0025” | | | |
| | ID PEEK, 8.5” Length (p/n 700009971)] coupled to a Xevo ® G2- | | | |
| | XS QTof Mass Spectrometer (available from Waters Technologies | | | |
| | Corporation, Milford, MA) | | | |
| Data Acquisition | MassLynx ™ 4.1 (available from Waters Technologies | | | |
| and Analysis: | Corporation, Milford, MA) | | | |
| Column: | Mixed Mode RPLC/Anion Exchange 1.7 μm, 2.1 × 150 mm | | | |
| Column Temperature: | 60° C. | | | |
| Seal Wash: | 10% HPLC grade Acetonitrile/90% HPLC grade water v/v (Seal | | | |
| | Wash interval set to 0.5 min) | | | |
| Sample Manager Wash: | HPLC grade water | | | |
| Mobile Phase A: | 18.2 MΩ HPLC grade water | | | |
| Mobile Phase B: | 100 mM Formic Acid, 100 mM Ammonium Formate in 40:60 | | | |
| | (v/v) water/acetonitrile | | | |
| Flow Rate: | 0.4 mL/min | | | |
| Gradient: | Time (min) | % A | % B | Curve |
| | 0.00 | 100.0 | 0.0 | 6 |
| | 36.00 | 78.0 | 22.0 | 6 |
| | 36.30 | 0.0 | 100.0 | 6 |
| | 37.30 | 0.0 | 100.0 | 6 |
| | 38.00 | 100.0 | 0.0 | 6 |
| | 45.00 | 100.0 | 0.0 | 6 |

| | |
|---|---|
| Sample Temperature | 8° C. |
| Samples: | 90 μL of RFMS-labeled released glycan (freshly prepared from 15 |
| | μg Orencia ®) |
| Sample dilution: | No dilution applied |
| Sample Injection Volume: | 1 μL |
| FLR Wavelength: | Excitation: 265 nm/Emission: 435 nm |
| FLR Scan Rate: | 10 points/second |
| Time Constant: | 0.2 second |
| MS Capillary Voltage: | 2.2 kV |
| Cone Voltage: | 75 V |
| Source Offset: | 80 V |
| Source Temperature: | 120° C. |
| Desolvation Temperature: | 500° C. |
| Desolvation Gas Flow Rate: | 600 L/Hr |
| Calibration | Sodium Iodide, 100-3000 m/z |
| Acquisition: | 700-3000 m/z |
| Scan time: | 0.1 second |

FIG. 6 shows the fluorescence (FLR) profile of RFMS-labeled glycans released from 5 mM tetramethylguanidine denatured Orencia® as obtained with a mixed mode, anion exchange reversed phase separation. Similar to intact analysis of deglycosylated protein, this tetramethylguanidine denatured sample exhibited a fluorescence chromatogram comparable to the positive control (FIG. 5D), indicating that the substituted guanidine denaturant is suitable for use with a RapiFluor-MS™ labeling strategy. That is, no significant interferences were observed in the LC-FLR-MS data.

### Example 4 (Prophetic Example): Use for Protein Denaturation and Subsequent Proteolytic Digestion

Prophetically speaking, a protein sample (50 μg) could be denatured with 50 mM tetramethylguanidine in a 50 mM Tris pH 7, 10 mM calcium chloride buffer by means of a 5 minute incubation at 90° C. The protein sample could then be cooled, optionally reduced and/or alkylated and desalted through sizing media, as can be done with a SizeX 100 desalting tip (available from Integrated Micro-Chromatography Systems, Inc (IMCS), Irmo, S.C.). The desalted sample could then be digested with trypsin in solution for 4 hours at 35° C. to 45° C. in a 50 mM Tris, 10 mM calcium chloride buffer or with an immobilized trypsin resin for 5 to 20 minutes at 50° C. to 70° C. Resulting peptide digest could then be analyzed by reversed phase chromatography and UV or mass spectrometric detection.

In another prophetic experiment, a protein sample could be incubated with 5 mM tetramethylguanidine at 90° C. in a 50 mM Tris pH 7, 10 mM calcium chloride buffer. Upon cooling, the denatured protein could optionally be reduced and/or alkylated and then be digested with in-solution trypsin for 4 hours at 35° C. to 45° C.

Alternatively, the denatured protein samples from either of the above procedures could be digested with immobilized protease at a temperature ranging from 45° C. to 75° C. for a time frame shorter than 4 hours.

### Example 5: Evaluation of Downstream Interference if Denaturants Used for Peptide Mapping

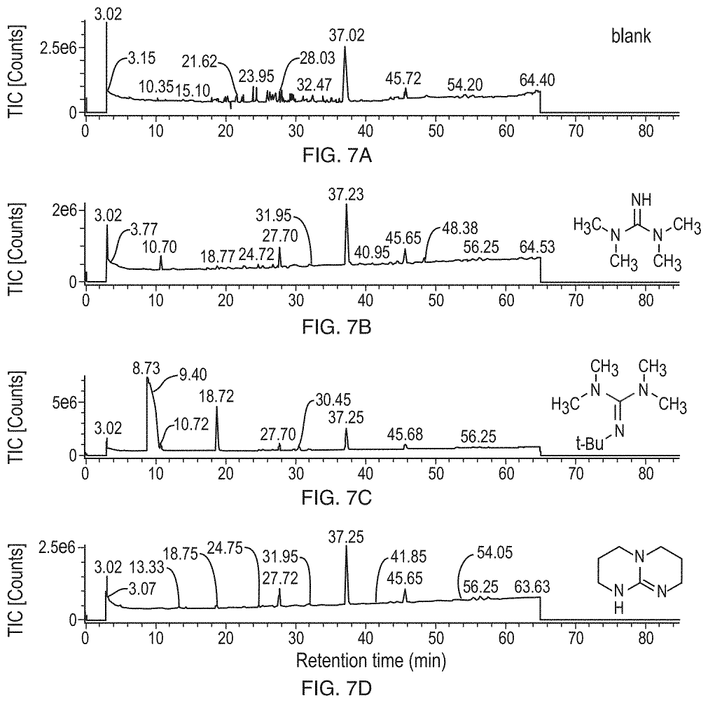

1 mM of each tetramethylguanidine denaturants (FIG. 7A is a blank; Reagent #1, FIG. 7B; Reagent #2, FIG. 7C; and Reagent #3, FIG. 7D) in digestion buffer were analyzed through a generic LC-MS method for peptide mapping. Shown in FIGS. 7B and 7D, reagents 1 and 3 did not appear to show significant interference peaks across the gradient (1-40% acetonitrile) where peptides normally elute. Reagent 2 in FIG. 7C showed two prominent peaks at retention time ˜10 min and 18.72 min. This could potentially introduce strong interference for peptides that co-elute in this time window, if reagent 2 is not removed from sample before LC-MS analysis. Not to be limited by theory, however, these interferences could be reduced or removed by utilizing higher grade of reagents. Table 3 shows the parameter settings for LCMS analysis on BioAccord™ (commercially available from Waters Technologies Corporation, Milford, Mass.).

TABLE 3
Parameter settings for LCMS analysis on BioAccord ™
ACQUITY ™ I-Class PLUS
Detection: ACUITY ™ Tunable UV (TUV) (available from Waters
Technologies Corporation, Milford, MA)
Column: ACUITY UPLC ™ BEH C18 column (p/n 186003555,
available from Waters Technologies Corporation, Milford, MA)
Column temp.: 65° C.
Sample temp.: 6° C.
Injection volume: 10 μL
Flow rate: 0.25 mL/min
Mobile phase A: 0.1% formic acid in H2O
Mobile phase B: 0.1% formic acid in acetonitrile
Gradient: 1% B over 5 min, 1%-40% B over 65 min, 15% B over 2 min
and 1% B for 14 min
ACQUITY RDa ™ Detector
MS system: ACQUITY RDa ™ Detector (available from Waters
Technologies Corporation, Milford, MA)
Ionization mode: ESI positive
Acquisition range: m/z 50-2000
Capillary voltage: 1.2 kV
Collision energy: 60-120 V
Cone voltage: 30 V
Desolvation energy: 350° C.
Intelligent data capture: on

While this disclosure has been particularly shown and described with reference to example embodiments thereof, it will be understood by those skilled in the art that various changes in form and details may be made therein without departing from the scope of the technology encompassed by the appended claims. For example, other chromatography systems or detection systems can be used.

1. A composition for protein denaturation, the composition comprising:
a non-nucleophilic denaturant comprising a substituted guanidine,
wherein the denaturant has a pKa value greater than about 10, and
wherein the concentration of the substituted guanidine is less than 250 mM.

2. (canceled)

3. The composition of claim 1, wherein the substituted guanidine comprises at least one from the group of tetramethylguanidine, tertbutyl tetramethylguanidine, triazabicyclodecene, or combinations thereof.

4. The composition of claim 3, wherein the substituted guanidine of tetramethylguanidine is 1,1,3,3-tetramethylguanidine with the chemical structure of

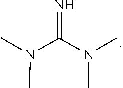

5. The composition of claim 3, wherein the substituted guanidine of tertbutyl tetramethylguanidine is 2-tert-butyl-1,1,3,3-tetramethylguanidine with the chemical structure of

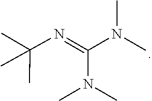

6. The composition of claim 3, wherein the substituted guanidine of triazabicyclodecene is 1,5,7-triazabicyclo[4.4.0]dec-5-ene with the chemical structure of

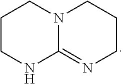

7. The composition of claim 3, wherein the substituted guanidine is a guanidinium cation.

8. The composition of claim 1, further comprising an additional denaturant of at least one from the group of sodium dodecylsulfate, n-lauryl sarcosine, lauric acid, cholic acid, or combinations thereof.

9. A composition for protein denaturation, the composition comprising:
a non-nucleophilic denaturant comprising a substituted amidine,
wherein the denaturant has a pKa value greater than about 10, and
wherein the concentration of the substituted amidine is less than 250 mM.

10. (canceled)

11. The composition of claim 9, wherein the substituted amidine comprises at least one from the group of hexanimidamide, acetamidine, propanimidamide, or combinations thereof.

12. A method of denaturing a sample comprising a protein, the method comprising:
incubating the sample with a non-nucleophilic denaturant, wherein the concentration of the denaturant is less than about 250 mM and wherein the denaturant has a pKa value greater than about 10;
heating the sample for a predetermined amount of time to denature the protein; and
cooling the sample to a reduced temperature.

13. The method of claim 12, wherein non-nucleophilic denaturant comprises substituted guanidine, substituted amidine, or a combination thereof.

14. The method of claim 13, wherein the substituted guanidine comprises tetramethylguanidine, tertbutyl tetramethylguanidine, triazabicyclodecene, or combinations thereof.

15. The method of claim 13, wherein the substituted amidine comprises hexanimidamide, acetamidine, propanimidamide, or combinations thereof.

16. The method of claim 12, wherein the denatured protein is unfolded and remains unfolded when the temperature is reduced to the reduced temperature.

17. (canceled)

18. (canceled)

19. The method of claim 12, wherein heating the sample comprises heating the sample to a temperature ranging from about 40° C. to about 100° C.

20. The method of claim 12, wherein the reduced temperature ranges from about 30° C. to 75° C.

21. The method of claim 12, further comprising diluting the cooled sample.

22. (canceled)

23. The method of claim 12, wherein digesting the sample comprises digesting the sample with a protease.

24. The method of claim 23, wherein the protease is trypsin, Lys-C, Arg-C, Glu-C, Asp-N, chymotrypsin, or combinations thereof.

25. The method of claim 12, further comprising treating the cooled sample with an endo or exoglycosidase.
